(19) United States
(12) Patent Application Publication
(10) Pub. No.: US 2024/0184202 A1
(43) Pub. Date: Jun. 6, 2024

(54) FUSED RING AROMATIC HYDROCARBON DERIVATIVE, PREPARATION METHOD THEREFOR, AND USE THEREOF IN LITHOGRAPHY

(71) Applicant: INSTITUTE OF CHEMISTRY, CHINESE ACADEMY OF SCIENCES, Beijing (CN)

(72) Inventors: Guoqiang YANG, Beijing (CN); Xue CONG, Beijing (CN); Xudong GUO, Beijing (CN); Rui HU, Beijing (CN); Shuangqing WANG, Beijing (CN)

(21) Appl. No.: 18/501,272

(22) Filed: Nov. 3, 2023

(30) Foreign Application Priority Data
Nov. 10, 2022 (CN) 202211408311.2

(51) Int. Cl. G03F 7/039; G03F 7/004

(52) U.S. Cl. CPC G03F 7/039; G03F 7/0045; G03F 7/0048

(57) ABSTRACT

A positive chemically amplified photoresist of a fused ring aromatic hydrocarbon derivative, a preparation method therefor, and use thereof are provided. A matrix component in the photoresist is a fused ring aromatic hydrocarbon derivative represented by general formula (I) that can be dissolved in organic solvents commonly used in photoresists. The photoresist composition can be prepared to give a uniform film, and a molecular glass serving as the matrix component does not precipitate particles in the film preparation process.

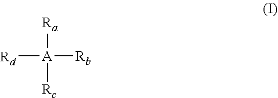

### TECHNICAL FIELD

The present disclosure belongs to the technical field of photoresists, and particularly relates to a fused ring aromatic hydrocarbon derivative, a preparation method therefor, and use thereof in lithography.

### BACKGROUND

Integrated circuits are the foundation of modern equipment manufacturing, and are obtained by large-scale and very large-scale integrated circuit manufacturing processes. With the rapid development of the semiconductor industry, electronic devices require integrated circuits (chips) with increasingly smaller dimensions and higher integration levels. Lithography technology is the most critical process in the manufacture of integrated circuits, and the wavelength used in the exposure process affects the resolution of integrated circuits. Since the 1980s, the lithography technology has been developed from the initial G-line (436 nm) and I-line (365 nm) lithography, to deep ultraviolet (248 nm and 193 nm) lithography, and then to extreme ultraviolet lithography (13.5 nm) of the next-generation lithography technology. The wavelength has decreased, and the minimum achievable dimensions of integrated circuits have become smaller. In addition, advanced lithography technologies such as nanoimprint lithography, electron beam lithography, and the like have been developed. The minimum feature dimension of integrated circuits has moved from the initial micron scale and the submicron scale to the nanometer scale.

Photoresists, as core materials in lithography, have also continuously developed and changed. Due to the large molecular weight and uneven distribution of traditional high-molecular chemically amplified photoresists, it is difficult to achieve a line pattern with high resolution and low line edge roughness. A molecular glass material is a small molecular organic compound with a relatively high glass transition temperature (Tg), has the characteristic of monodispersity of molecular weight, is in an amorphous state, has a very high melting point and thermal stability, and can meet the requirements of EUV lithography after the introduction of an acid-sensitive group. In the traditional chemically amplified photoresists, the relatively small molecular weight of the host materials can cause low contrast of the photoresists and poor steepness of the stripes, and thus it is necessary to develop a molecular glass photoresist with a relatively large molecular weight. An etching process is a main process of patterning treatment associated with lithography, which selectively removes materials uncovered by a photoresist on the surface of a silicon wafer via a chemical or physical method, so that the photoresist is required to have etching resistance, and the etching resistance of the photoresist can be improved by increasing the content of benzene rings (fused rings) in the host material structure. The present disclosure develops a novel molecular glass photoresist, which is expected to achieve a positive chemically amplified photoresist with higher resolution and contrast as well as high etching resistance.

### SUMMARY

The object of the present disclosure is to provide a fused ring aromatic hydrocarbon derivative and a preparation method therefor.

Another object of the present disclosure is to provide use of a plurality of fused ring aromatic hydrocarbon derivatives described above in lithography and a positive photoresist composition.

The present disclosure provides a compound represented by formula (I):

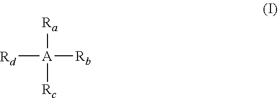

wherein A is selected from a fused ring aromatic hydrocarbon;
Rₐ, R_{b}, R_{c}, and R_{d} are the same or different and are each independently selected from H or

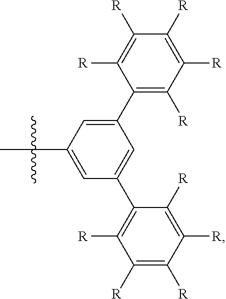

provided that at least one of Rₐ, R_{b}, R_{c}, and R_{d} is

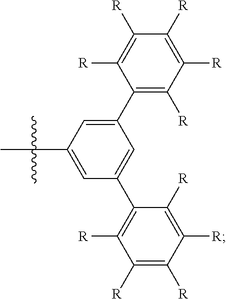

the

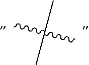

is a linking site;
each R is the same or different and is independently selected from H, C₁₋₂₀ alkyl, or an acid-sensitive group, provided that at least one R is selected from an acid-sensitive group.

According to an embodiment of the present disclosure, the fused ring aromatic hydrocarbon is selected from C₉₋₄₀ aromatic hydrocarbon, preferably C₁₀₋₁₆ aromatic hydrocarbon.

According to an embodiment of the present disclosure, the fused ring aromatic hydrocarbon is selected from naphthalene, anthracene, phenanthrene, or pyrene.

According to an embodiment of the present disclosure, Rₐ, R_{b}, R_{c}, and R_{d} are the same or different and are each independently selected from H or

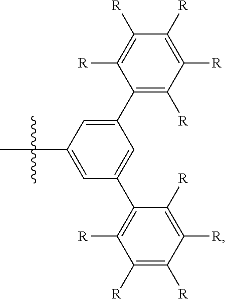

provided that one, two, three, or four of Rₐ, R_{b}, R_{c}, and R_{d} are

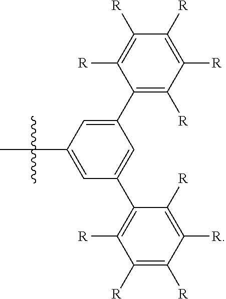

According to an embodiment of the present disclosure, Rₐ, R_{b}, R_{c}, and R_{d} are selected from

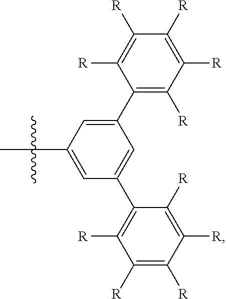

wherein in the

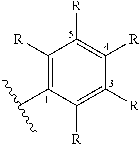

group, when there is only one R, it is preferably linked at position 4; when there are two R, they are preferably linked at positions 3 and 4, or positions 4 and 5; when there are three R, they are preferably linked at positions 3, 4, and 5.

According to an embodiment of the present disclosure, each R is the same or different and is independently selected from H, C₁₋₆ alkyl, or an acid-sensitive group, provided that at least one R is selected from an acid-sensitive group.

According to an embodiment of the present disclosure, the acid-sensitive group is —OR₂; R₂ is selected from —COOC₁₋₂₀ alkyl, —COC₁₋₂₀ alkyl, —COC₃₋₂₀ cycloalkyl, and —(CH₂)_{q}—COOC₃₋₂₀ cycloalkyl, q being an integer from 0 to 6; or the C₁₋₂₀ alkyl and C₃₋₂₀ cycloalkyl are further optionally substituted with one, two, or more halogens or C₁₋₂₀ alkyl.

According to an embodiment of the present disclosure, the acid-sensitive group is —OR₂; R₂ is selected from —COOC₁₋₆ alkyl, —COC₁₋₆ alkyl, —COC₃₋₁₂ cycloalkyl, and —(CH₂)_{q}—COOC₃₋₁₂ cycloalkyl, q being selected from 0, 1, 2, or 3; or the C₁₋₆ alkyl and C₃₋₁₂ cycloalkyl are further optionally substituted with one, two, or more C₁₋₆ alkyl.

According to a preferred embodiment of the present disclosure, the acid-sensitive group is selected from the following structures:

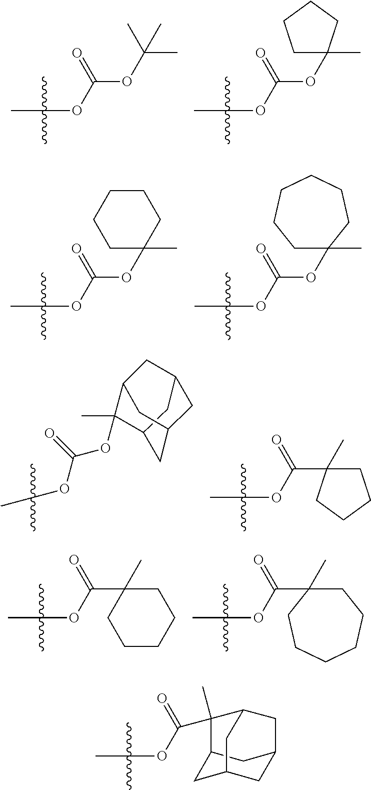

the

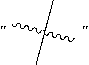

is a linking site.

According to an embodiment of the present disclosure, the compound represented by formula (I) preferably has a structure represented by formula (A) or formula (B):

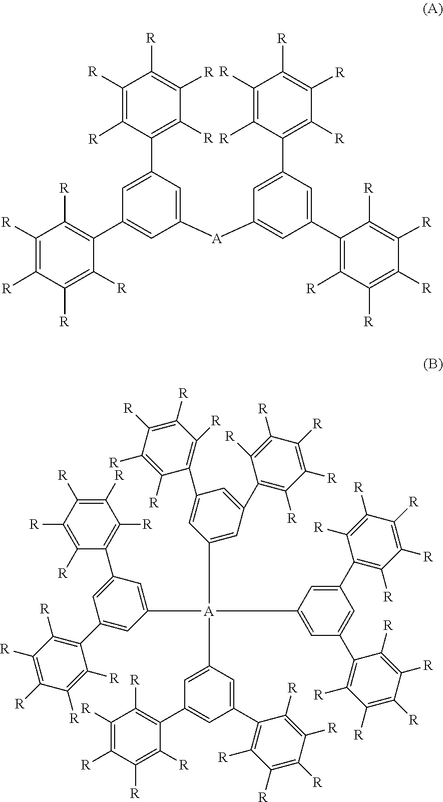

wherein R and A have the definitions described above.

According to a preferred embodiment of the present disclosure, the compound represented by formula (I) is selected from the following structures:

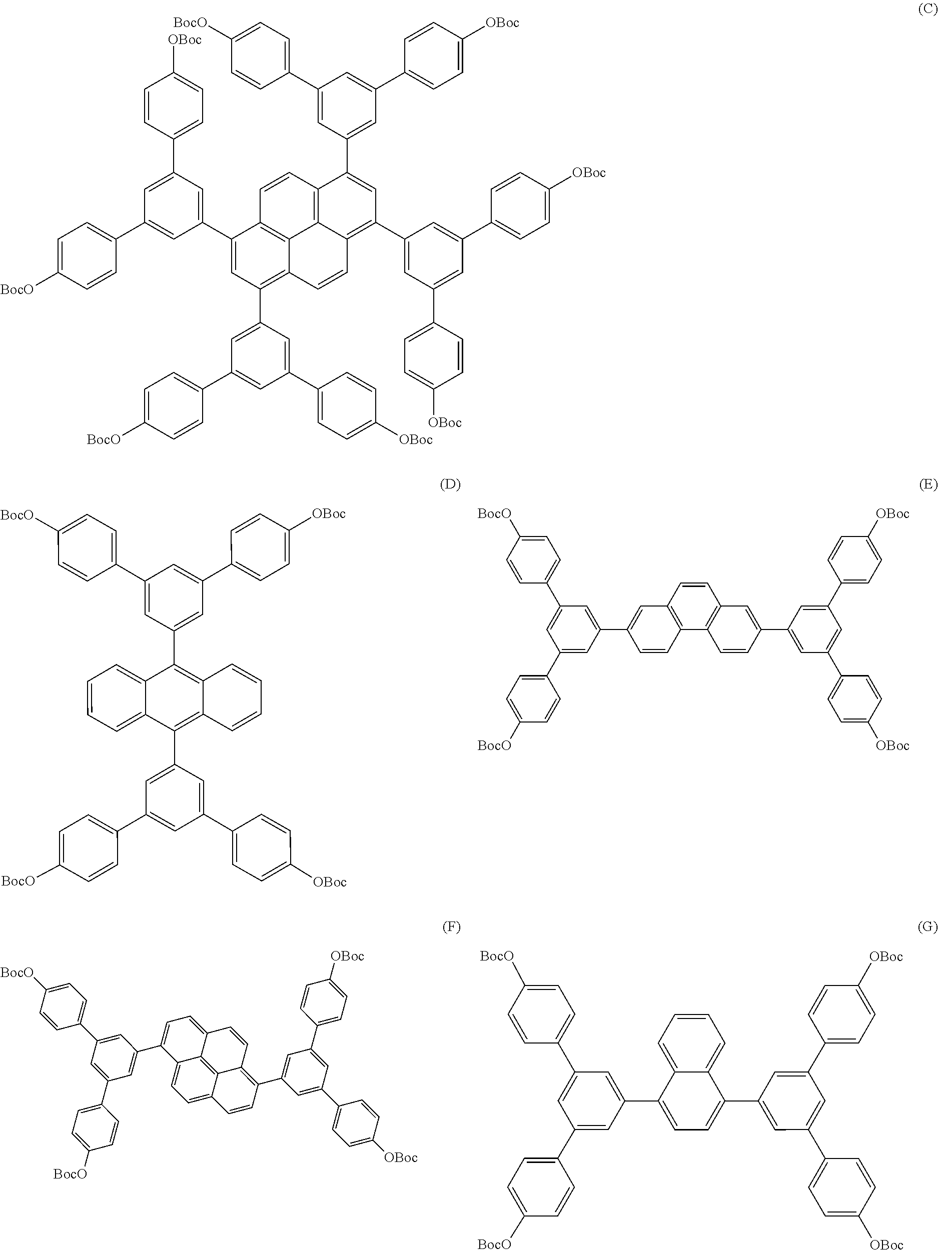

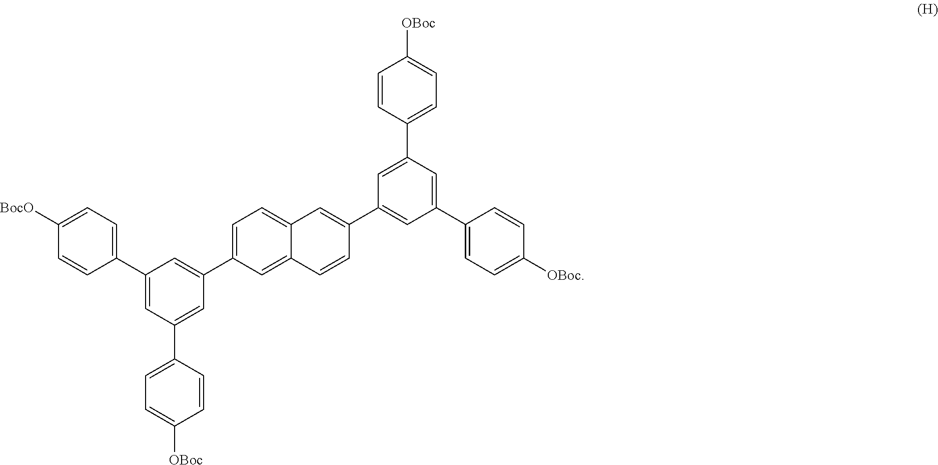

The present disclosure further provides a preparation method for the compound represented by formula

(I), comprising the following steps:

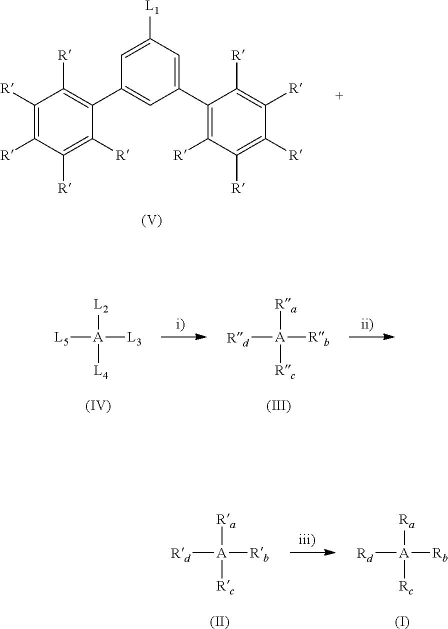

(i) reacting a compound represented by formula (V) with a compound represented by formula (IV) to give a compound represented by formula (III);
(ii) reacting the compound represented by formula (III) to give a compound represented by formula (II); and
(iii) reacting the compound represented by formula (II) with a compound providing an acid-sensitive group to give the compound represented by formula (I);
   wherein A, Rₐ, R_{b}, R_{c}, and R_{d} have the definitions described above;
R′ₐ, R′_{b}, R′_{c}, and R′_{d} are selected from

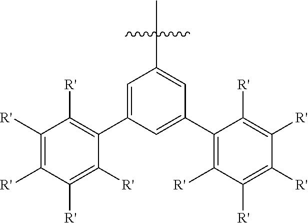

or H, provided that not all are H; each R′ is the same or different and is independently selected from OH or H, provided that not all are H;
R″ₐ, R″_{b}, R″_{c}, and R″_{d} are selected from

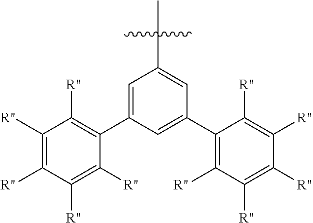

or H, provided that not all are H; each R″ is the same or different and is independently selected from H, -C₁₋₂₀ alkyl, and —OC₁₋₂₀ alkyl, provided that at least one R″ is —OC₁₋₂₀ alkyl;

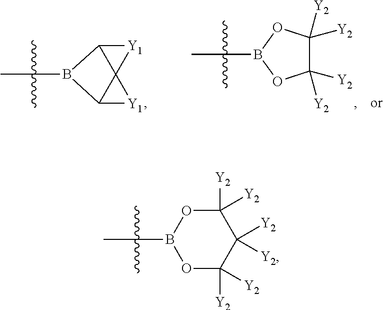

L₁ is —B(OH)₂, —B(OC₁₋₂₀ alkyl)₂, wherein Y₁ is C₁₋₂₀ alkylene, and Y₂ is each independently selected from —H or C₁₋₂₀ alkyl; L₂, L₃, L₄, and L₅ are the same or different and are each independently selected from halogen or H, provided that not all are H;
the compound providing the acid-sensitive group is selected from an acid-sensitive group-containing acid anhydride or a compound composed of a leaving group L₆ and an acid-sensitive group; L₆ is a leaving group, such as F, Cl, Br, or I.

According to an embodiment of the present disclosure, the reaction in step i) is heated to reflux in an organic solvent, wherein the organic solvent is, for example, 1,4-dioxane or tetrahydrofuran.

According to an embodiment of the present disclosure, the reaction in step i) is performed in the presence of a basic reagent, for example, in the presence of sodium carbonate.

According to an embodiment of the present disclosure, the reaction in step i) is performed in the presence of a palladium-containing catalyst, for example, in the presence of tetrakis(triphenylphosphine)palladium(0).

According to an embodiment of the present disclosure, the reaction in step i) is performed in an inert gas atmosphere, for example, in argon.

According to an embodiment of the present disclosure, the reaction in step ii) is performed in the presence of a Lewis acid, for example, in boron tribromide.

According to an embodiment of the present disclosure, the reaction in step iii) is performed in the presence of a deacid reagent, for example, in the presence of 4-dimethylaminopyridine (DMAP).

According to an embodiment of the present disclosure, the reaction in step iii) is performed in an inert gas atmosphere, for example, in argon.

According to an embodiment of the present disclosure, a compound represented by formula (C-4) is prepared using the following method:

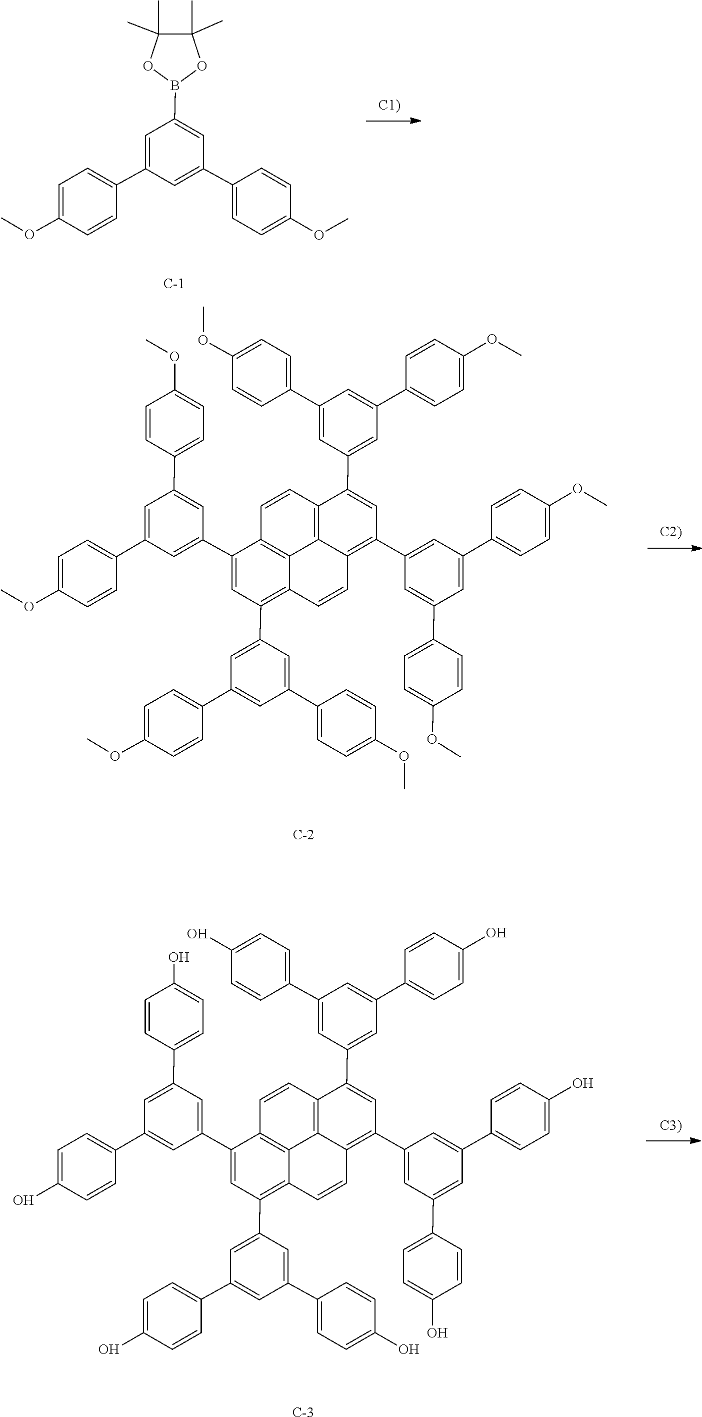

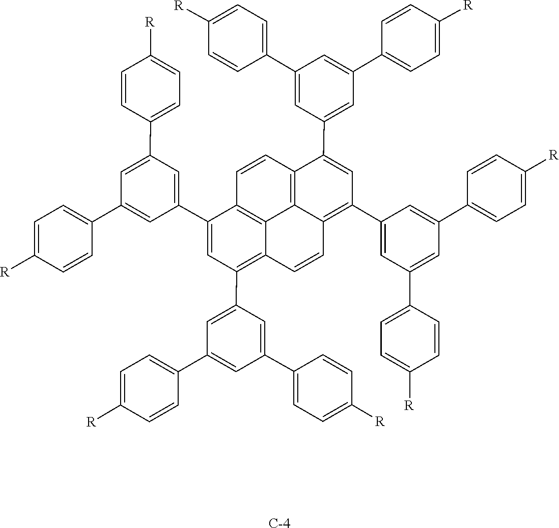

(c1) reacting a compound represented by formula (C-1) with 1,3,6,8-tetrabromopyrene to give a compound represented by formula (C-2);
(c2) reacting the compound represented by formula (C-2) to give a compound represented by formula (C-3); and
(c3) reacting the compound represented by formula (C-3) with an acid-sensitive group-containing acid anhydride or a compound composed of a leaving group L₆ and an acid-sensitive group to give the compound represented by formula (C-4);
wherein R and L₆ have the definitions described above.

According to an embodiment of the present disclosure, a compound represented by formula (D-4) is prepared using the following method:

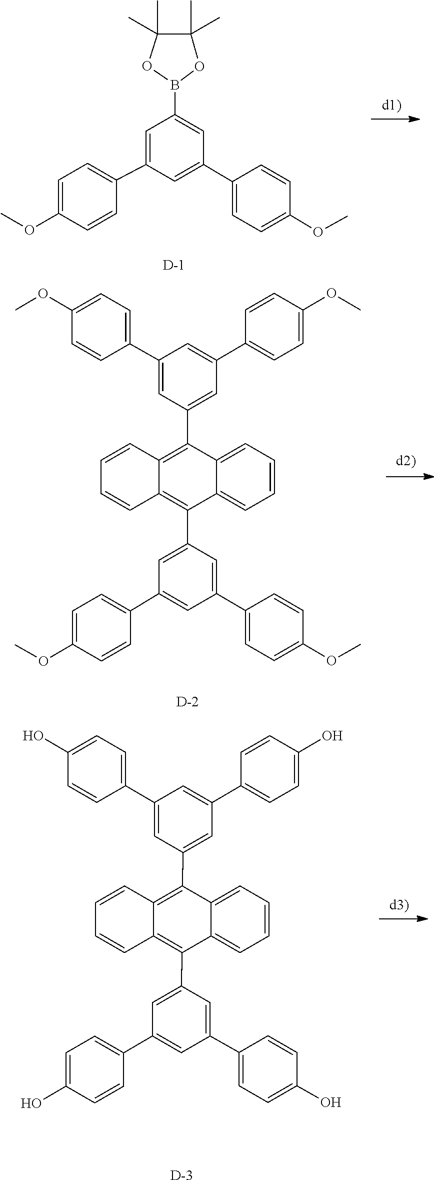

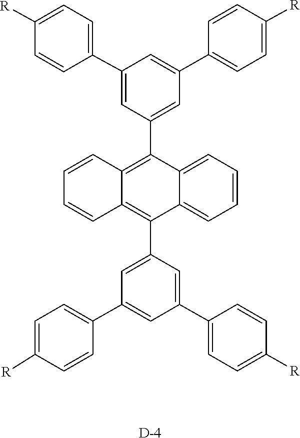

(d1) reacting a compound represented by formula (D-1) with 9,10-dibromoanthracene to give a compound represented by formula (D-2);
(d2) reacting the compound represented by formula (D-2) to give a compound represented by formula (D-3); and
(d3) reacting the compound represented by formula (D-3) with an acid-sensitive group-containing acid anhydride or a compound composed of a leaving group L₆ and an acid-sensitive group to give the compound represented by formula (D-4);
wherein R and L₆ have the definitions described above.

The present disclosure further provides use of the compound represented by formula (I) in lithography, such as use thereof in a photoresist, preferably use thereof in the preparation of a positive photoresist. The present disclosure further provides a positive photoresist composition comprising a matrix, wherein the matrix is selected from at least one of compounds represented by formula (I).

According to an embodiment of the present disclosure, the composition further comprises a photoacid generator, wherein the photoacid generator is, for example, selected from bis(trichloromethyl)-s-triazine derivatives, onium salt compounds, sultone compounds, and sulfonate compounds; preferably, the photoacid generator used is at least one of the following:

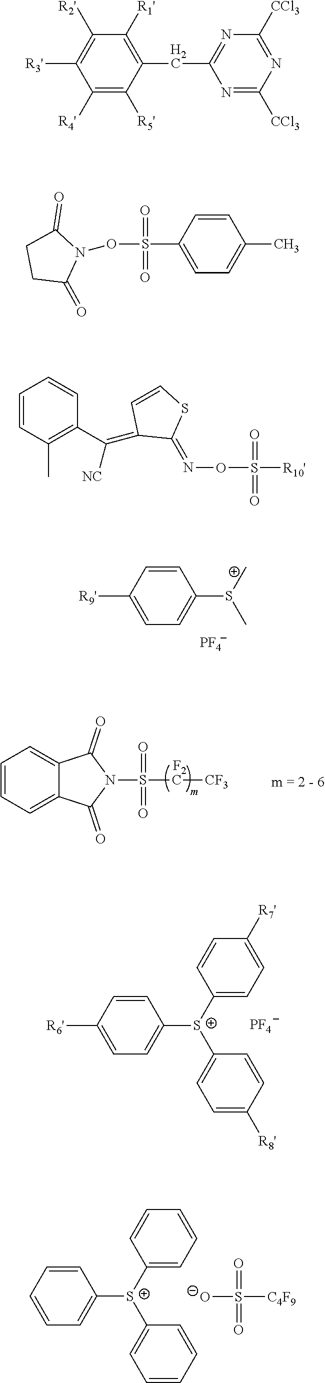

wherein are the same or different and are each independently selected from H, C₁₋₂₀ alkyl, an ester group, acylamino, carboxyl, an aldehyde group, and hydroxyl.

According to an embodiment of the present disclosure, the photoacid generator is selected from

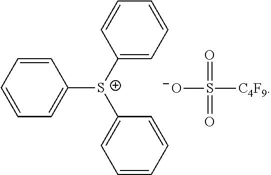

According to an embodiment of the present disclosure, the composition further comprises an organic base, wherein the organic base is, for example, selected from various nitrogen-containing organic amine compounds, such as at least one of methylamine, dimethylamine, trimethylamine, triethylamine, tripropylamine, tributylamine, trioctylamine, hexanediamine, benzylamine, and cyclohexyl amine.

According to an embodiment of the present disclosure, the composition further comprises an organic solvent, wherein the organic solvent is, for example, selected from alkane, ester, ether, and haloalkane compounds; the organic solvent is preferably at least one of 1,2,3-trichloropropane, anisole, propylene glycol methyl ether acetate, propylene glycol monoacetate, propylene glycol diacetate, ethyl lactate, propylene glycol monomethyl ether, methyl ethyl ketone, methyl isobutyl ketone, neopentyl acetate, butyl acetate, diethylene glycol ethyl ether, dichloromethane, and tetrahydrofuran.

According to an embodiment of the present disclosure, in the photoresist composition, the mass of the matrix accounts for 2%-30%, preferably 4%-20%, of the total mass of the positive photoresist composition.

According to an embodiment of the present disclosure, in the photoresist composition, the mass of the photoacid generator accounts for 2%-30%, preferably 5%-20%, of the mass of the matrix.

According to an embodiment of the present disclosure, in the photoresist composition, the mass of the organic base accounts for 0.02%-8% of the mass of the matrix.

According to an embodiment of the present disclosure, in the photoresist composition, the mass of the organic solvent accounts for 70%-96% of the total mass of the photoresist composition.

According to an embodiment of the present disclosure, the photoresist composition further comprises other additives, such as sensitizers, surfactants, dyes, stabilizers, and the like.

The present disclosure further provides use of the photoresist composition in deep ultraviolet (248 nm and 193 nm) lithography, extreme ultraviolet (13.5 nm, EUV) lithography, nanoimprint lithography (NIL), and electron beam lithography (EBL).

### Beneficial Effects

The matrix component in the positive photoresist composition of the present disclosure takes a fused ring aromatic hydrocarbon represented by formula (I) as a central core structure, so the positive photoresist composition has a relatively high melting point, can meet the requirements of lithography technology, has a stable structure, and has no change of a film structure in high-temperature baking. The matrix component in the positive photoresist composition of the present disclosure is a three-dimensional asymmetric and amorphous small molecular compound that can be dissolved in organic solvents commonly used in photoresists. The photoresist composition of the present disclosure can be prepared to give a uniform film, and a molecular glass compound serving as a matrix component is not precipitated in the film preparation process. Therefore, the film prepared from the photoresist composition of the present disclosure has good resolution, photosensitivity, and adhesion, and is easy to store.

### BRIEF DESCRIPTION OF THE DRAWINGS

FIG. 1 is a diagram showing a thermogravimetric analysis test of compound C prepared in Example 1.

FIG. 2 is a diagram showing an X-ray diffraction test of the compound C prepared in Example 1.

FIG. 3 is an electron micrograph of a deep ultraviolet lithographic pattern of a photoresist composition prepared in Example 3.

FIG. 4 is an electron micrograph of an electron beam lithographic pattern of the photoresist composition prepared in Example 3.

FIG. 5 is an electron micrograph of an electron beam lithographic pattern of a photoresist composition prepared in Example 4.

### DEFINITIONS AND DESCRIPTION

Unless otherwise defined, all technical and scientific terms used herein have the same meaning as commonly understood by those skilled in the art to which the subject matter of the claims belong.

“More” refers to three or more.

The term “halogen” includes F, Cl, Br, or I.

The term “C₁₋₂₀ alkyl” should be understood to refer to a linear or branched saturated monovalent hydrocarbyl group having 1-20 carbon atoms, preferably “C₁₋₆ alkyl”. “C₁₋₆ alkyl” refers to linear and branched alkyl groups having 1, 2, 3, 4, 5, or 6 carbon atoms. The alkyl is, for example, methyl, ethyl, propyl, butyl, pentyl, hexyl, isopropyl, isobutyl, sec-butyl, tent-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, etc., or isomers thereof.

The term “C₃₋₂₀ cycloalkyl” should be understood to refer to a saturated monovalent monocyclic, bicyclic, or polycyclic hydrocarbon ring (also known as fused hydrocarbon ring) having 3-20 carbon atoms. The bicyclic or polycyclic cycloalkyl includes ortho-fused cycloalkyl, bridged cycloalkyl, and spirocycloalkyl; the ortho-fused ring refers to a fused ring structure formed by two or more ring structures sharing two adjacent ring atoms (i.e., sharing a bond) with each other. The bridged ring refers to a fused ring structure formed by two or more ring structures sharing two non-adjacent ring atoms with each other. The spiro ring refers to a fused ring structure formed by two or more ring structures sharing one ring atom with each other. For example, the C₃₋₂₀ cycloalkyl may be C₃₋₈ monocyclic cycloalkyl such as cyclopropyl, cyclobutyl, cyclopentyl, cyclohexyl, cycloheptyl, and cyclooctyl, or may be C₇₋₁₂ ortho-fused cycloalkyl such as a decahydronaphthalene ring, or may also be C₇₋₁₂ bridged cycloalkyl such as norbornane, adamantane, and bicyclo[2,2,2]octane.

The term “C₉₋₄₀ aromatic hydrocarbon” should be understood to preferably refer to an aromatic or partially aromatic fused ring having 9-40 carbon atoms, which may be a single aromatic ring or multiple aromatic rings fused together, preferably “C₉₋₂₀ aromatic hydrocarbon”. The term “C₉₋₂₀ aromatic hydrocarbon” should be understood to preferably refer to an aromatic or partly aromatic fused ring having 9, 10, 11, 12, 13, 14, 15, 16, 17, 18, 19, or 20 carbon atoms, particularly a ring having 10-16 carbon atoms (“C₁₀₋₁₆ aromatic hydrocarbon”), for example, a ring having 9 carbon atoms (“C₉ aromatic hydrocarbon”) such as indan or indene, or a ring having 10 carbon atoms (“C₁₀ aromatic hydrocarbon”) such as tetrahydronaphthalene, dihydronaphthalene, or naphthalene, or a ring having 13 carbon atoms (“C₁₃ aromatic hydrocarbon”) such as fluorene, or a ring having 14 carbon atoms (“C₁₄ aromatic hydrocarbon”) such as anthracene, or a ring having 16 carbon atoms (“C₁₆ aromatic hydrocarbon”) such as pyrene. When the C₉₋₄₀ aromatic hydrocarbon is substituted, it may be monosubstituted or polysubstituted. In addition, the substitution site is not limited, and may be, for example, ortho-substitution, para-substitution, or meta-substitution.

### DETAILED DESCRIPTION

The embodiments of the present disclosure will be further illustrated in detail with reference to the following specific examples. It will be appreciated that the following examples are merely exemplary illustrations and explanations of the present disclosure, and should not be construed as limiting the protection scope of the present disclosure. All techniques implemented based on the content of the present disclosure described above are included within the protection scope of the present disclosure. Unless otherwise stated, the starting materials and reagents used in the following examples are all commercially available products or can be prepared using known methods.

### Example 1: Preparation of Compound C

### Synthetic Route of Compound C:

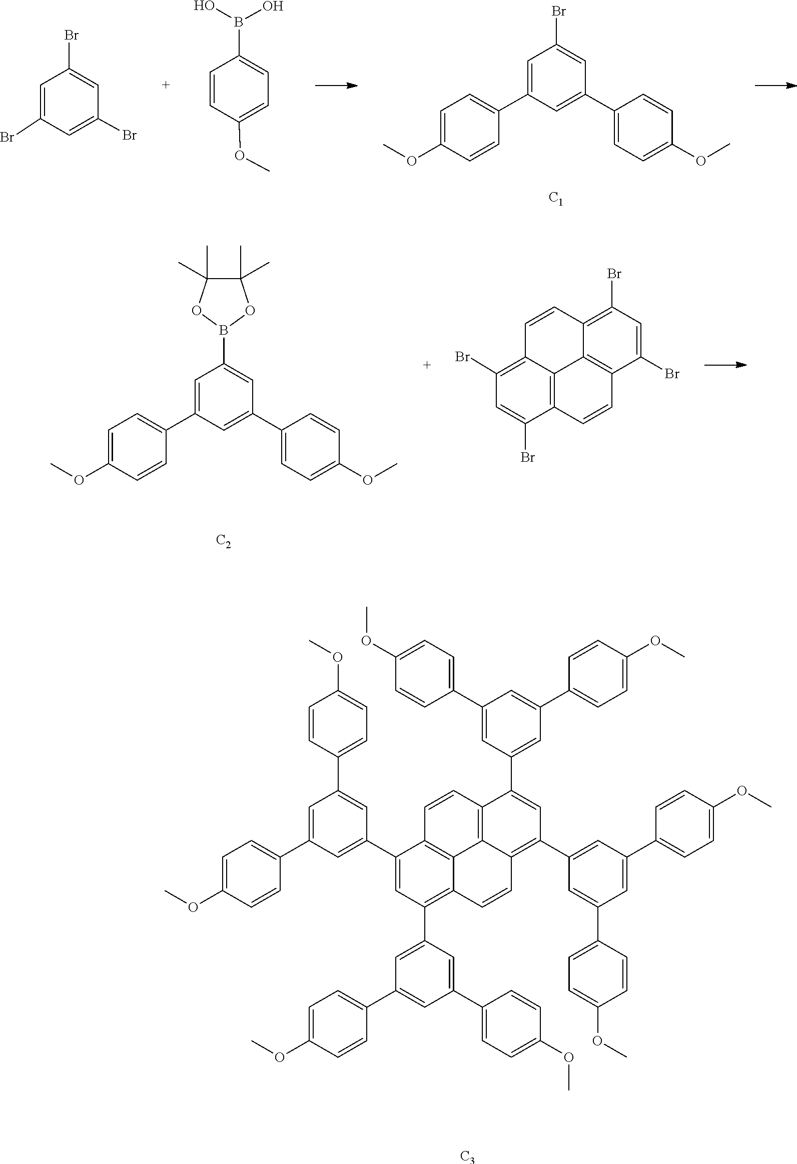

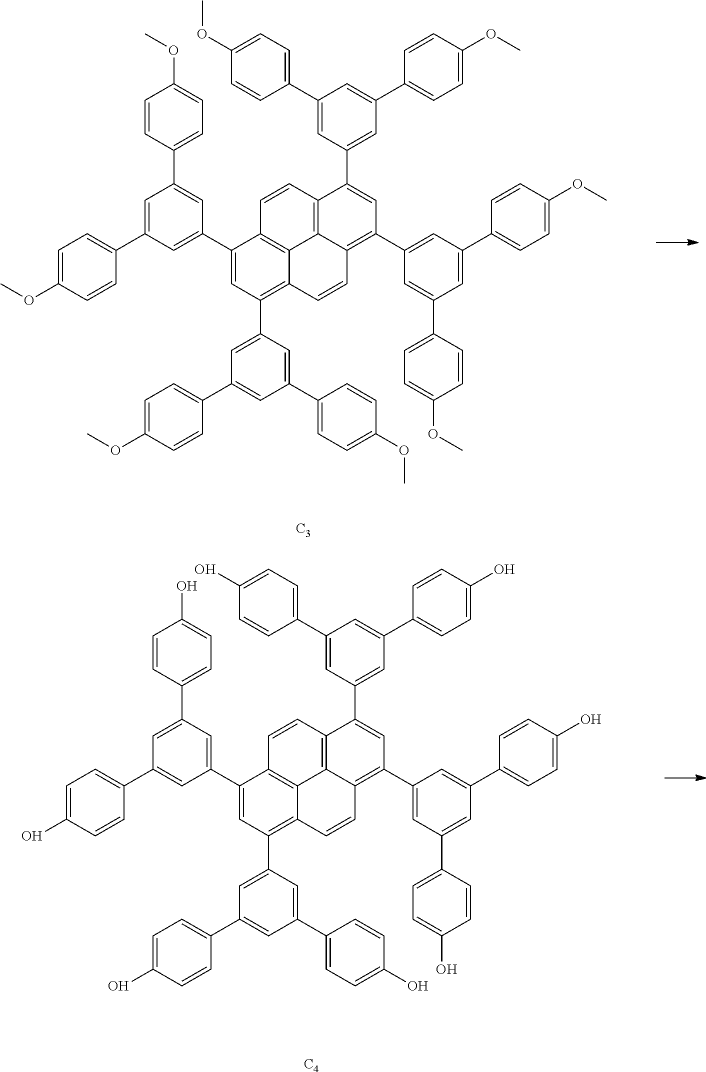

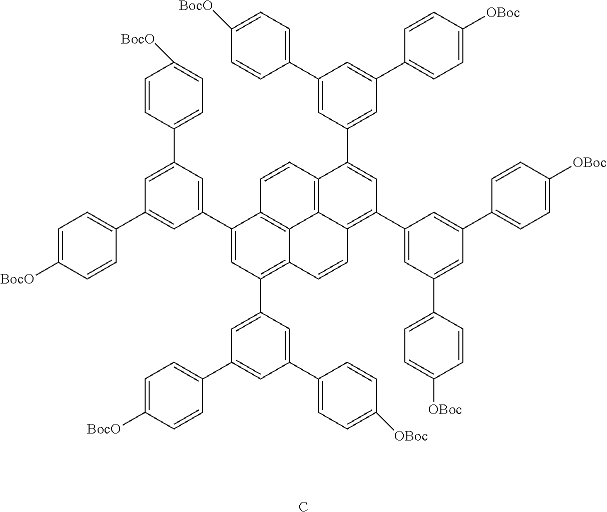

### Synthesis of Compound C₁:

15.2 g of 4-methoxyphenylboronic acid, 0.92 g of tetrakis(triphenylphosphine)palladium(0), 17 g of Na₂CO₃, and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 150 mL of 1,4-dioxane and 100 mL of ultrapure water were injected into the three-necked flask. 12.6 g of 1,3,5-tribromobenzene was dissolved in 50 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation and separated by column chromatography to give compound C₁ (6.49 g).

### Synthesis of Compound C₂:

4.57 g of bis(pinacolato)diboron, 4.42 g of KOAc, 327 mg of Pd(dppf)Cl₂, and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 35 mL of 1,4-dioxane was injected into the three-necked flask. 5.54 g of compound C₁ was dissolved in 30 mL of 1,4-dioxane, and the solution was injected into the dropping funnel. The reaction system was heated to 80° C. and stirred, the solution in the dropping funnel was added dropwise, and the reaction mixture was reacted for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound C₂ (5.71 g).

### Synthesis of Compound C₃:

1.04 g of 1,3,6,8-tetrabromopyrene, 2.21 g of Na₂CO₃, 0.277 g of tetrakis(triphenylphosphine)palladium(0), and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 10 mL of 1,4-dioxane and 8 mL of ultrapure water were injected into the three-necked flask. 4.16 g of the compound C₂ was dissolved in 40 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation and separated by column chromatography to give compound C₃ (1.63 g).

### Synthesis of Compound C₄:

1.36 g of the compound C₃ was dissolved in 70 mL of dichloromethane, and then the mixture was added to a three-necked flask with a magnetic stir bar (the three-necked flask was connected to a gas-guide tube, a constant pressure dropping funnel, and a rubber septum, respectively). The system was placed in an ice-water bath, and 4.64 mL of BBr₃ was injected into the dropping funnel. The reaction system was stirred. After the slow dropwise addition was completed, the reaction system was cooled to room temperature and reacted for 24 h. After the reaction was completed, the reaction liquid was transferred into another constant pressure dropping funnel connected to a three-necked flask. 100 mL of ice water was added to the three-necked flask, and the reaction liquid was slowly added dropwise in an ice-water bath and stirred for 2 h. After the reaction was completed, the reaction liquid was washed with a large amount of saturated brine and ethyl acetate, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound C₄ (1.24 g).

### Synthesis of Compound C:

1.24 g of the compound C₄ and 97.6 mg of 4-dimethylaminopyridine (DMAP) were placed in a three-necked flask (the three-necked flask was connected to a gas-guide tube, a constant pressure dropping funnel, and a rubber septum, respectively). 10 mL of THF was injected into the three-necked flask. A compound Boc anhydride (2.62 g) was dissolved in 10 mL of THF, and the solution was injected into the dropping funnel. The reaction system was placed in an ice-water bath and stirred. The solution in the dropping funnel was added dropwise. After the dropwise addition was completed, the reaction system was slowly cooled to room temperature and reacted for 12 h. After the reaction was completed, the reaction liquid was concentrated by rotary evaporation, washed with a large amount of saturated brine and ethyl acetate, dried over anhydrous Na₂SO₄ for 1 h, and filtered to give a filtrate. The filtrate was concentrated by rotary evaporation and separated by column chromatography to give compound C (1.0 g).

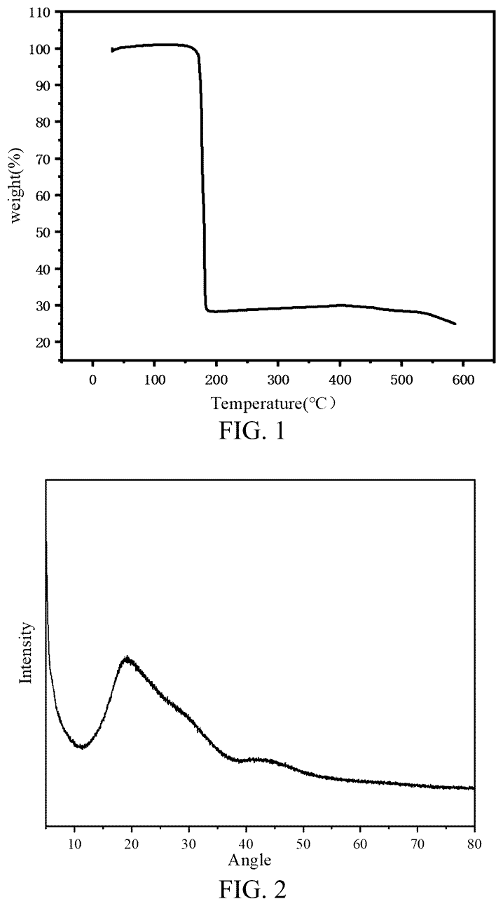

A thermogravimetric analysis of the compound C is shown in FIG. 1, and the decomposition temperature is about 180° C.; an XRD analysis is shown in FIG. 2, indicating that the compound C is an amorphous compound and does not crystallize.

¹H NMR (400 MHz, DMSO) δ8.47 (d, J=7.7 Hz, 6H), 8.05 (d, J=21.2 Hz, 12H), 7.97 (d, J=8.5 Hz, 16H), 7.33 (d, J=8.5 Hz, 16H), 1.54 (s,72H). HRMS(MALDI): calculated [M+H]⁺: 2043.84; found: 2043.834.

### Example 2: Preparation of Compound F

### Synthetic Route of Compound F:

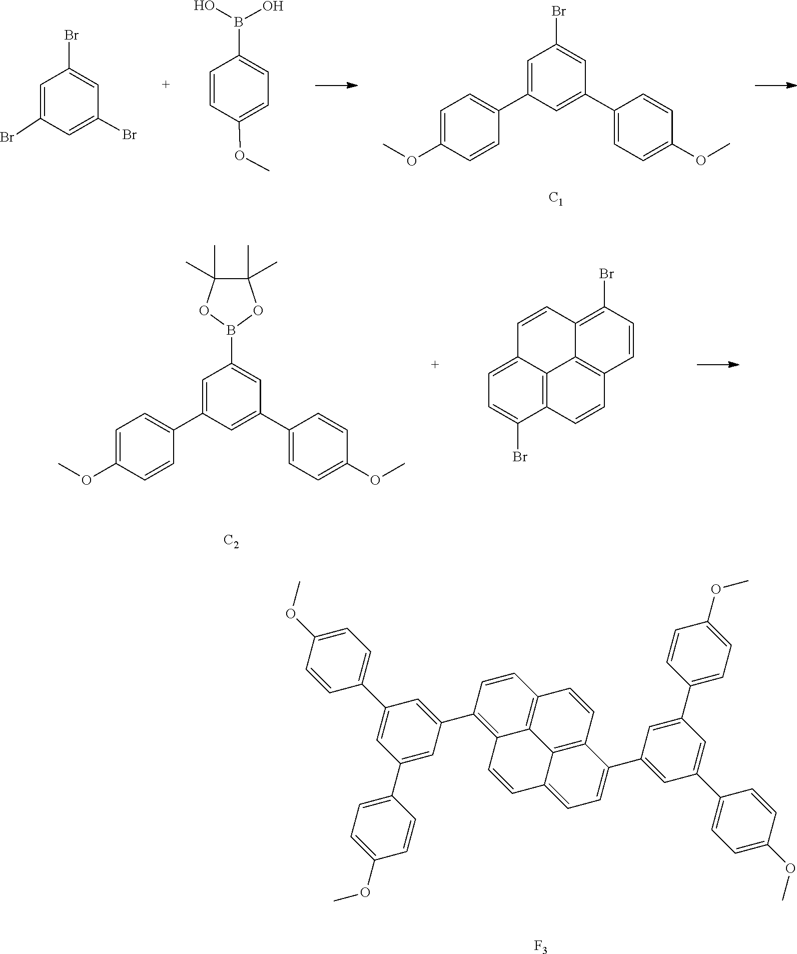

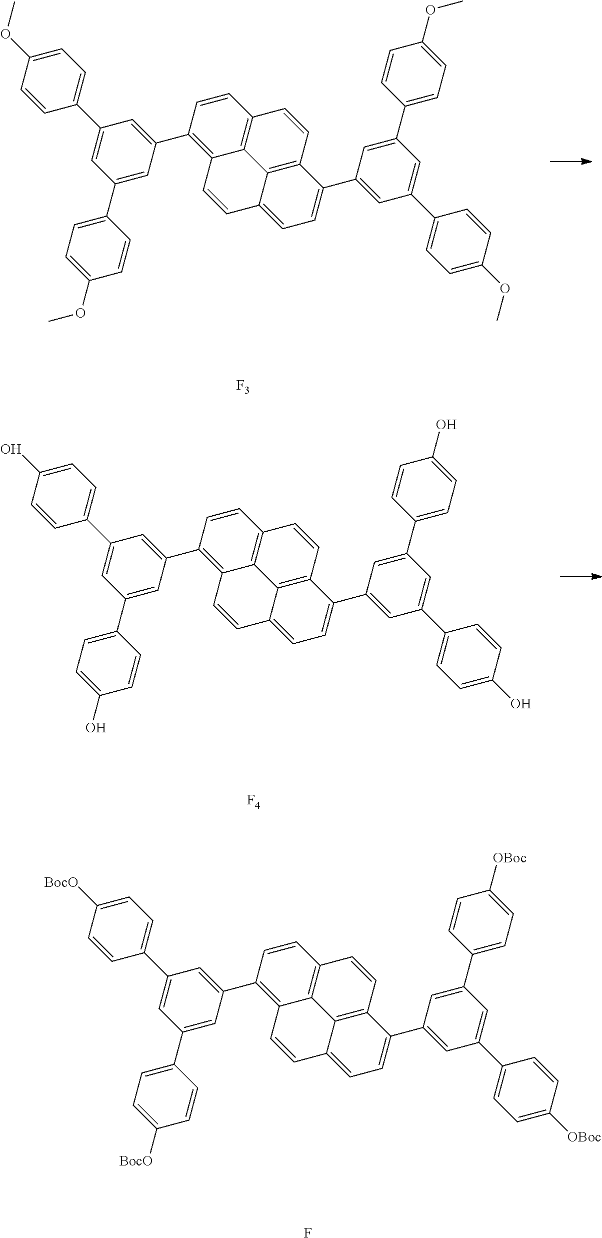

### Synthesis of Compound F₃:

1.08 g of 1,6-dibromopyrene, 2.86 g of Na₂CO₃, 0.208 g of tetrakis(triphenylphosphine)palladium(0), and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 10 mL of 1,4-dioxane and 15 mL of ultrapure water were injected into the three-necked flask. 3.12 g of the compound C₂ was dissolved in 20 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation, a large amount of petroleum ether was added for precipitation, sonication was performed, and the reaction liquid was filtered to give compound F₃ (2.34 g).

### Synthesis of Compound F₄:

2.34 g of the compound F₃ was dissolved in 30 mL of dichloromethane, and then the mixture was added to a three-necked flask with a magnetic stir bar (the three-necked flask was connected to a gas-guide tube, a constant pressure dropping funnel, and a rubber septum, respectively). The system was placed in an ice-water bath, and 1.74 mL of BBr₃ was injected into the dropping funnel. The reaction system was stirred. After the slow dropwise addition was completed, the reaction system was cooled to room temperature and reacted for 24 h. After the reaction was completed, the reaction liquid was transferred into another constant pressure dropping funnel connected to a three-necked flask. 50 mL of ice water was added to the three-necked flask, and the reaction liquid was slowly added dropwise in an ice-water bath and stirred for 2 h. After the reaction was completed, the reaction liquid was washed with a large amount of saturated brine and ethyl acetate, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound F₄ (2.16 g).

### Synthesis of Compound F:

723 mg of the compound F₄ and 4.89 mg of 4-dimethylaminopyridine (DMAP) were placed in a three-necked flask (the three-necked flask was connected to a gas-guide tube, a constant pressure dropping funnel, and a rubber septum, respectively). 10 mL of THF was injected into the three-necked flask. A compound Boc anhydride (1.31 g) was dissolved in 5 mL of THF, and the solution was injected into the dropping funnel. The reaction system was placed in an ice-water bath and stirred. The solution in the dropping funnel was added dropwise. After the dropwise addition was completed, the reaction system was slowly cooled to room temperature and reacted for 12 h. After the reaction was completed, the reaction liquid was concentrated by rotary evaporation, washed with a large amount of saturated brine and ethyl acetate, dried over anhydrous Na₂SO₄ for 1 h, and filtered to give a filtrate. The filtrate was concentrated by rotary evaporation and separated by column chromatography to give compound F (724 mg). ¹H NMR (300 MHz, CDCl₃) δ8.29 (dd, J=15.3, 8.6 Hz, 4H), 8.09 (d, J=7.7 Hz, 4H), 7.80 (dd, J=31.8, 8.7 Hz, 14H), 7.29 (t, J=8.3 Hz, 8H), 1.58 (s, 36H). HRMS(MALDI): calculated [M+H]⁺: 1122.46; found: 1122.45.

### Example 3: Photoresist Composition Comprising Compound C

Photoresist composition:
(1) matrix:

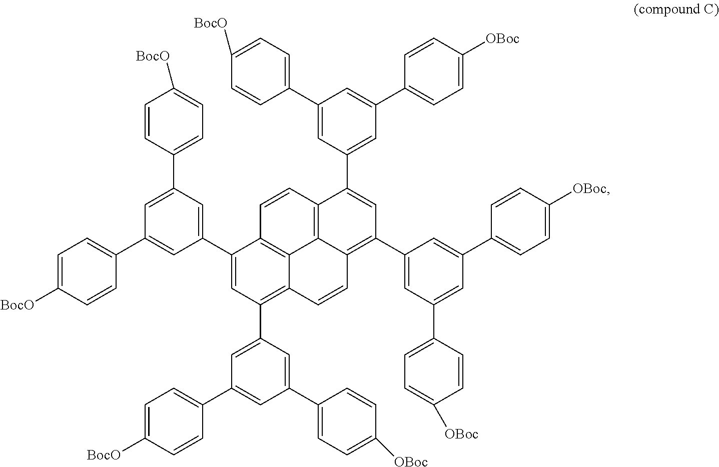

50 mg;
(2) photoacid generator:

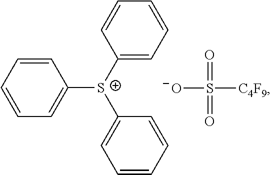

2.5 mg;
(3) organic base: trioctylamine, 0.125 mg; and
(4) organic solvent: propylene glycol methyl ether acetate (PGMEA), 2 mL.

### Example 4: Photoresist Composition Comprising Compound F

Photoresist composition:
(1) matrix:

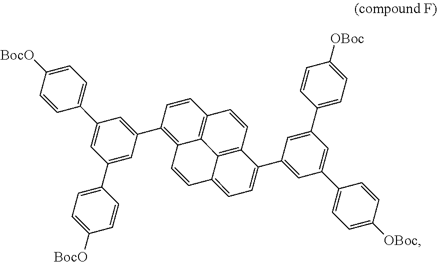

50 mg;
(2) photoacid generator:

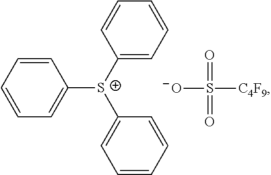

2.5 mg;
(3) organic base: trioctylamine, 0.125 mg; and
(4) organic solvent: propylene glycol methyl ether acetate (PGMEA), 2 mL.

### Example 5: Photoresist Composition Comprising Compound D

Photoresist composition:
(1) matrix:

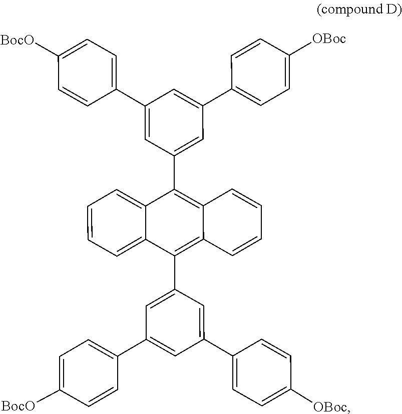

50 mg;
(2) photoacid generator:

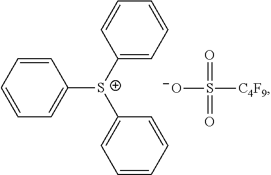

2.5 mg;
(3) organic base: trioctylamine, 0.125 mg; and
(4) organic solvent: propylene glycol methyl ether acetate (PGMEA), 2 mL.

Among them, the compound D was prepared by methods similar to those of Examples 1 and 2. HRMS(MALDI) [M+H]⁺: 1098.45.

### Example 6: Photoresist Composition Comprising Compound G

Photoresist composition:
(1) matrix:

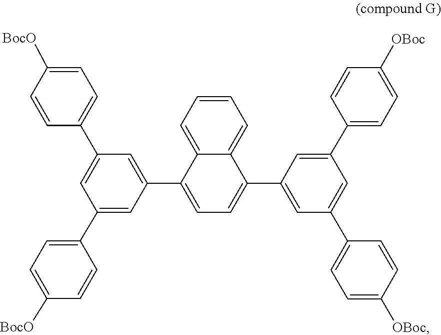

50 mg;
(2) photoacid generator:

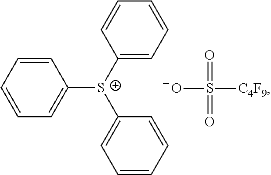

2.5 mg;
(3) organic base: trioctylamine, 0.125 mg; and
(4) organic solvent: propylene glycol methyl ether acetate (PGMEA), 2 mL.

Among them, the compound G was prepared by methods similar to those of Examples 1 and 2. HRMS(MALDI) [M+H]⁺: 1048.43.

### Test Example 1

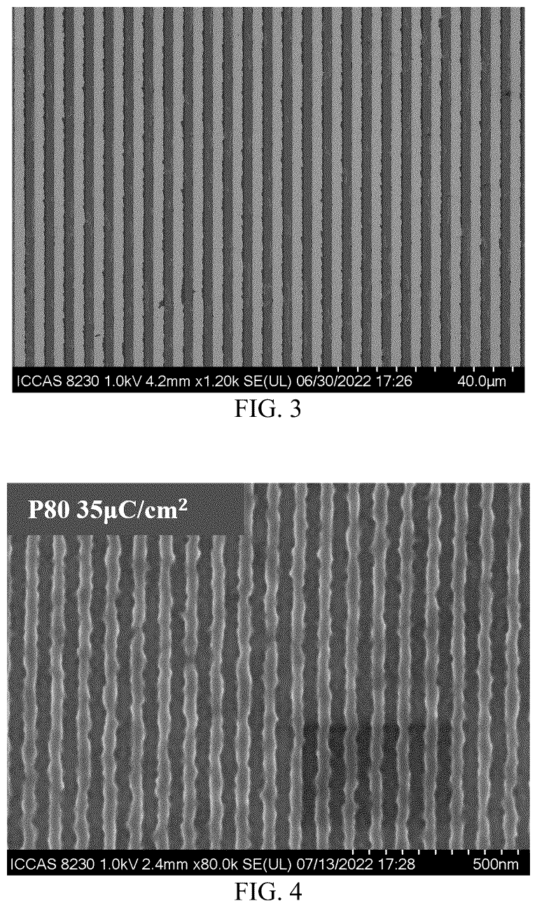

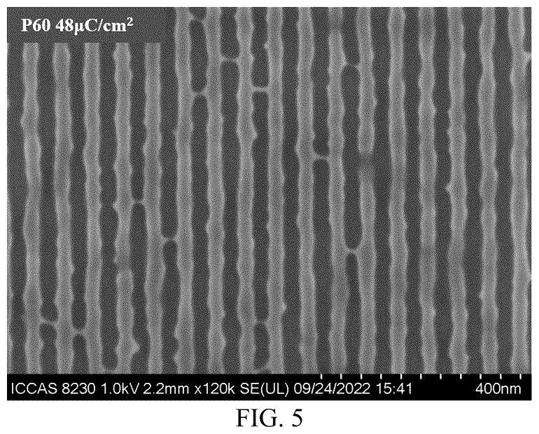

The photoresist composition comprising compound C in Example 3 was adopted, and PGMEA was used as a solvent. A 50-100 nm photoresist film was obtained by spin coating a silicon wafer. The photoresist composition had good film-forming performance, and the obtained film had uniform thickness. The deep ultraviolet electron beam lithography was performed to give a micron-scale lithographic pattern, as shown in FIG. 3; the electron beam lithography was performed in the National Center for Nanoscience and Technology, and a line lithographic pattern with a period of 80 nm and a line width of 40 nm was obtained at a dosage of 35 μC/cm², as shown in FIG. 4, indicating that the photoresist composition prepared by the compound of the present disclosure has good resolution and photosensitivity.

### Test Example 2

The photoresist composition comprising compound F in Example 4 was adopted, and PGMEA was used as a solvent. A 50-100 nm photoresist film was obtained by spin coating a silicon wafer. The photoresist composition had good film-forming performance, and the obtained film had uniform thickness. The electron beam lithography was performed in the National Center for Nanoscience and Technology, and a line lithographic pattern with a period of 60 nm and a line width of 30 nm was obtained, as shown in FIG. 5.

The embodiments of the technical solutions of the present disclosure have been described above by way of example. It should be understood that the protection scope of the present disclosure is not limited to the embodiments described above. Any modification, equivalent replacement, improvement, and the like made by those skilled in the art without departing from the spirit and principle of the present disclosure shall fall within the protection scope of the claims of the present application.

1. A compound represented by formula (I): 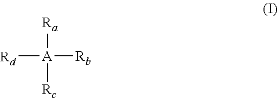
wherein A is selected from a fused ring aromatic hydrocarbon;
Rₐ, R_{b}, R_{c}, and R_{d} are the same or different and are each independently selected from H or 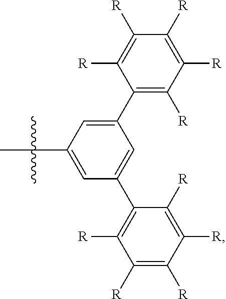
provided that at least one of Rₐ, R_{b}, R_{c}, and R_{d} is 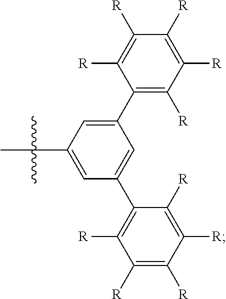
the 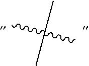
is a linking site;
each R is the same or different and is independently selected from H, C₁₋₂₀ alkyl, or an acid-sensitive group, provided that at least one R is selected from an acid-sensitive group.

2. The compound according to claim 1, wherein the fused ring aromatic hydrocarbon is selected from C₉₋₄₀ aromatic hydrocarbon, preferably C₁₀₋₁₆ aromatic hydrocarbon;
preferably, the fused ring aromatic hydrocarbon is selected from naphthalene, anthracene, phenanthrene, or pyrene.

3. The compound according to claim 1, wherein Rₐ, R_{b}, R_{c}, and R_{d} are the same or different and are each independently selected from H or 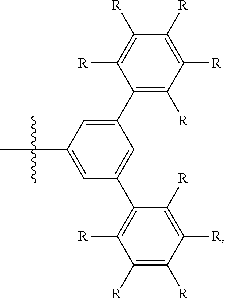
provided that one, two, three, or four of Rₐ, R_{b}, R_{c}, and R_{d} are 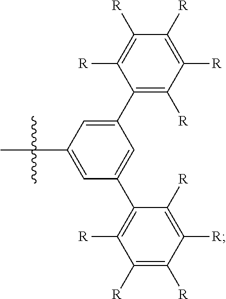
preferably, Rₐ, R_{b}, R_{c}, and R_{d} are selected from 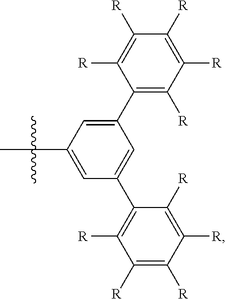
wherein in the group, 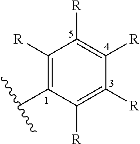
when there is only one R, it is preferably linked at position 4; when there are two R, they are preferably linked at positions 3 and 4, or positions 4 and 5; when there are three R, they are preferably linked at positions 3, 4, and 5;
preferably, each R is the same or different and is independently selected from H, C₁₋₆ alkyl, or an acid-sensitive group, provided that at least one R is selected from an acid-sensitive group;
preferably, the acid-sensitive group is —OR₂; R₂ is selected from —COOC₁₋₂₀ alkyl, —COC₁₋₂₀ alkyl, —COC₃₋₂₀ cycloalkyl, and —(CH₂)_{q}—COOC₃₋₂₀ cycloalkyl, q being an integer from 0 to 6; or the C₁₋₂₀ alkyl and C₃₋₂₀ cycloalkyl are further optionally substituted with one, two, or more halogens or C₁₋₂₀ alkyl;
preferably, the acid-sensitive group is —OR₂; R₂ is selected from —COOC₁₋₆ alkyl, —COC₁₋₆ alkyl, —COC₃₋₁₂ cycloalkyl, and —(CH₂)_{q}—COOC₃₋₁₂ cycloalkyl, q being selected from 0, 1, 2, or 3; or the C₁₋₆ alkyl and C₃₋₁₂ cycloalkyl are further optionally substituted with one, two, or more C₁₋₆ alkyl;
preferably, the acid-sensitive group is selected from the following structures: 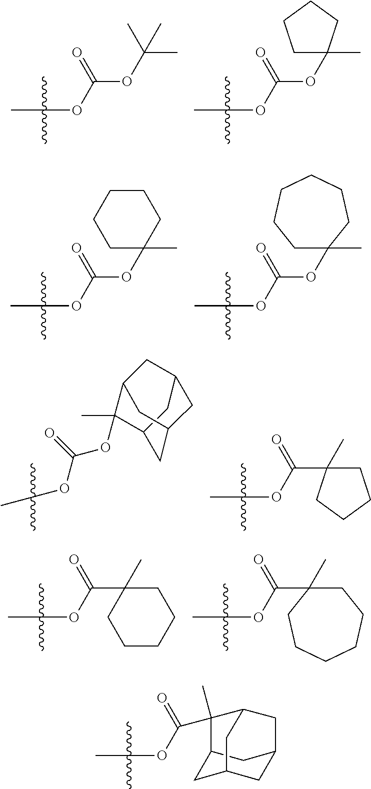
the 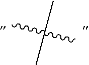
is a linking site.

4. The compound according to claim 1, wherein the compound has a structure represented by formula (A) or formula (B): 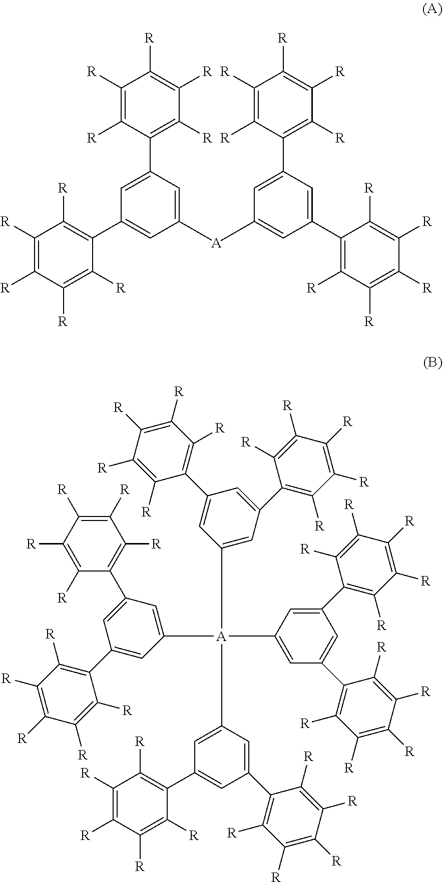
wherein R and A have the definitions described in claim 1.

5. The compound according to claim 1, wherein the compound is selected from the following structures:

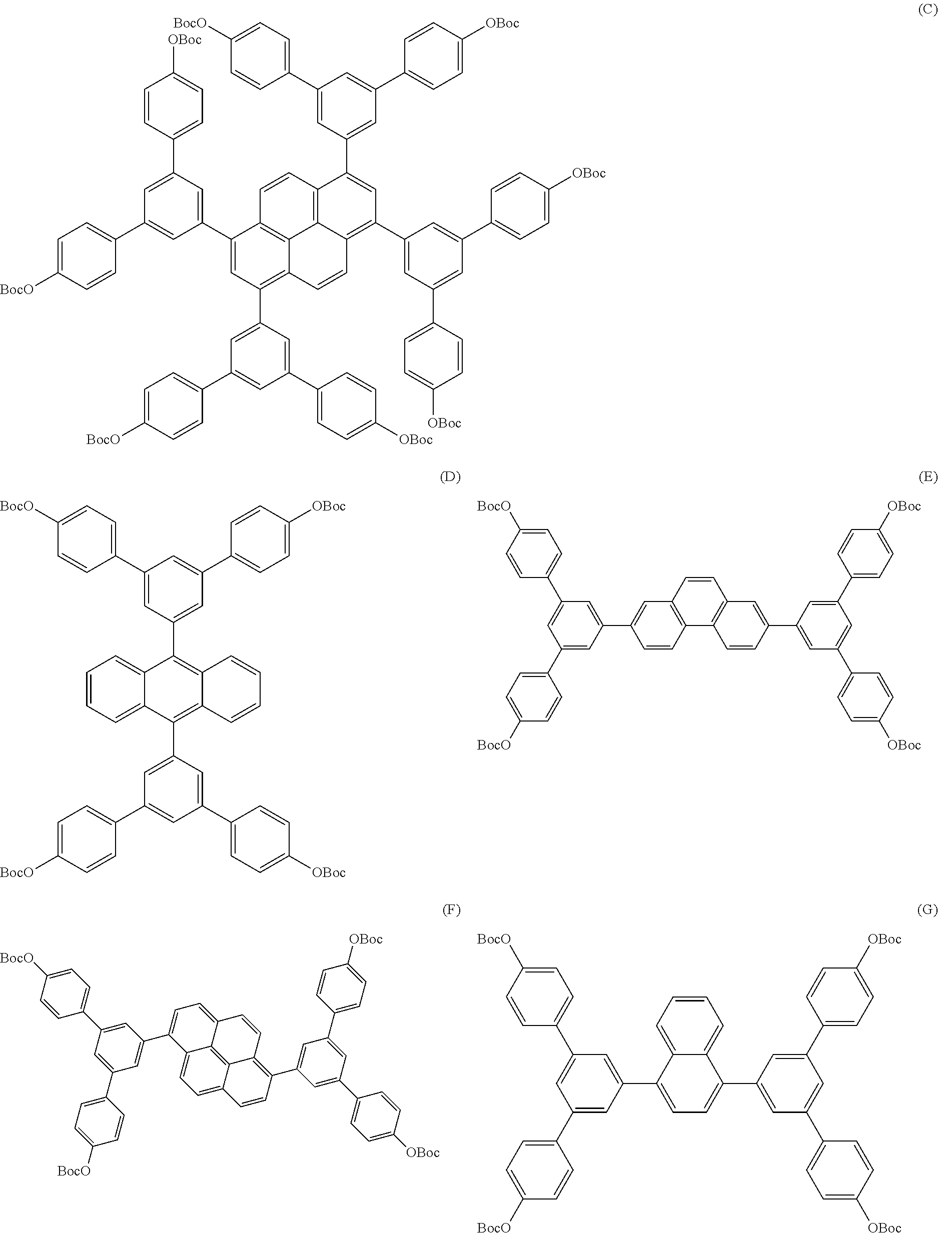

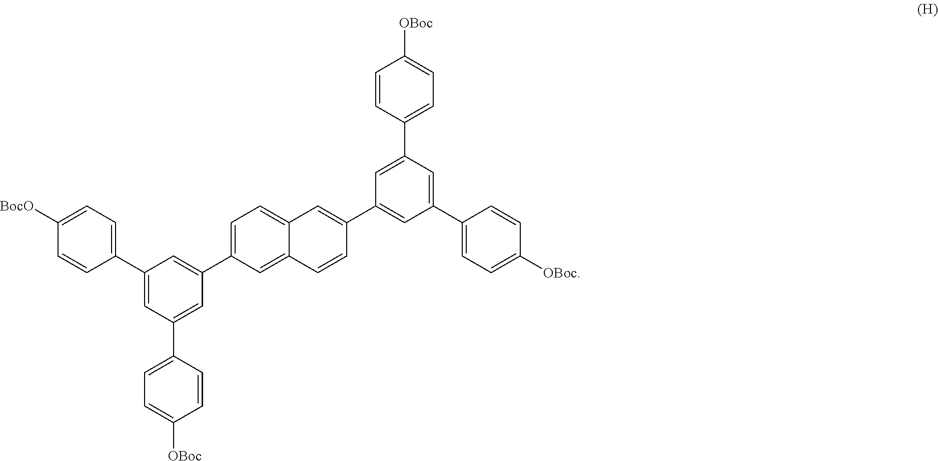

6. A preparation method for the compound according to claim 1, comprising the following steps: 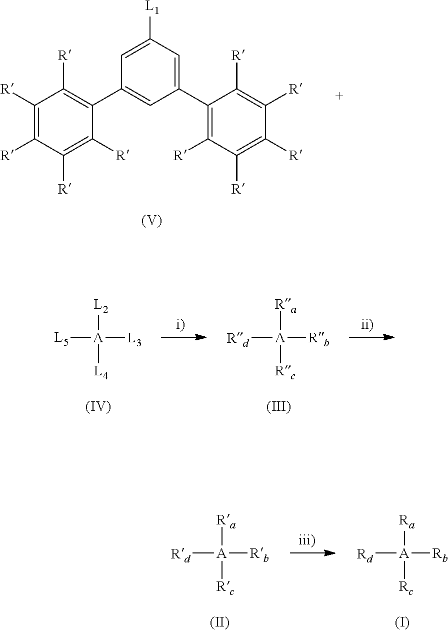
(i) reacting a compound represented by formula (V) with a compound represented by formula (IV) to give a compound represented by formula (III);
(ii) reacting the compound represented by formula (III) to give a compound represented by formula (II); and
(iii) reacting the compound represented by formula (II) with a compound providing an acid-sensitive group to give the compound represented by formula (I);
wherein A, Rₐ, R_{b}, R_{c}, and R_{d} have the definitions described in claim 1;
R′ₐ, R′_{b}, R′_{c}, and R′_{d} are selected from 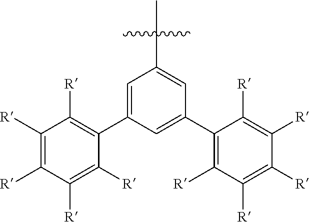
or H, provided that not all are H; each R′ is the same or different and is independently selected from OH or H, provided that not all are H;
R″ₐ, R″_{b}, R″_{c}, and R″_{d} are selected from 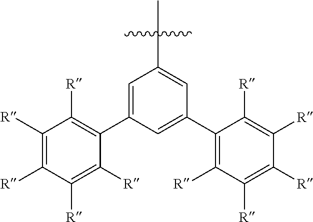
or H, provided that not all are H;
each R″ is the same or different and is independently selected from H, -C₁₋₂₀ alkyl, and —OC₁₋₂₀ alkyl, provided that at least one R is —OC₁₋₂₀ alkyl;
L₁ is —B(OH)₂, —B(OC₁₋₂₀ alkyl)₂, 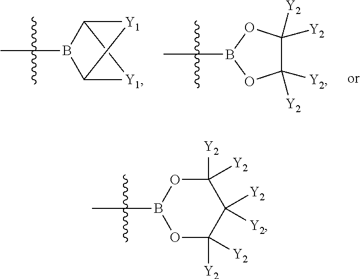
wherein Y₁ is C₁₋₂₀ alkylene, and Y₂ is each independently selected from —H or C₁₋₂₀ alkyl; L₂, L₃, L₄, and L₅ are the same or different and are each independently selected from halogen or H, provided that not all are H;
the compound providing the acid-sensitive group is selected from an acid-sensitive group-containing acid anhydride or a compound composed of a leaving group L₆ and an acid-sensitive group; L₆ is a leaving group, such as F, Cl, Br, or I.

7. Use of the compound according to claim 1 in lithography, such as use thereof in a photoresist, preferably use thereof in the preparation of a positive photoresist.

8. A positive photoresist composition, comprising a matrix, wherein the matrix is selected from at least one of compounds represented by formula (I);
preferably, the composition further comprises a photoacid generator, wherein the photoacid generator is, for example, selected from bis(trichloromethyl)-s-triazine derivatives, onium salt compounds, sultone compounds, and sulfonate compounds; preferably, the photoacid generator is at least one of the following: 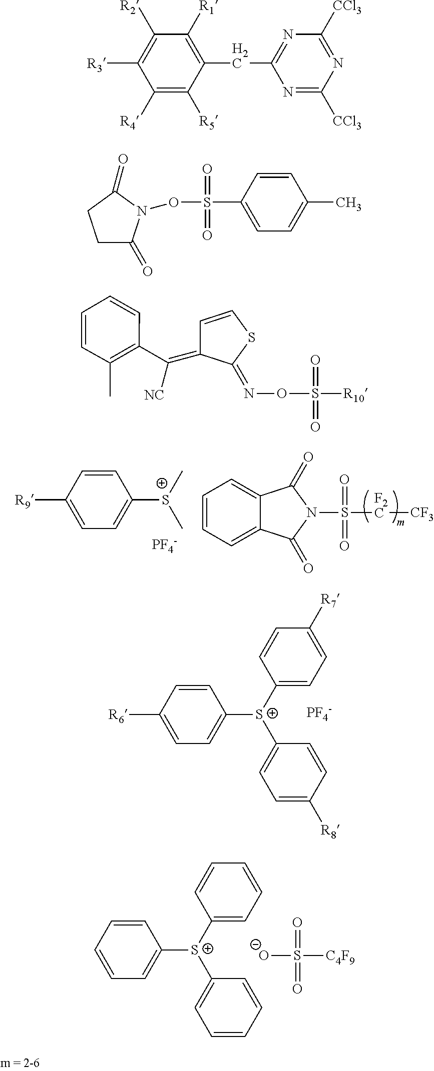
wherein are the same or different and are each independently selected from H, C₁₋₂₀ alkyl, an ester group, acylamino, carboxyl, an aldehyde group, and hydroxyl;
preferably, the photoacid generator is selected from 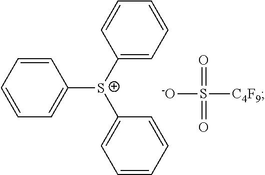
preferably, the composition further comprises an organic base, wherein the organic base is, for example, selected from various nitrogen-containing organic amine compounds, such as at least one of methylamine, dimethylamine, trimethylamine, triethylamine, tripropylamine, tributylamine, trioctylamine, hexanediamine, benzylamine, and cyclohexylamine;
preferably, the composition further comprises an organic solvent, wherein the organic solvent is, for example, selected from alkane, ester, ether, and haloalkane compounds; the organic solvent is preferably at least one of 1,2,3-trichloropropane, anisole, propylene glycol methyl ether acetate, propylene glycol monoacetate, propylene glycol diacetate, ethyl lactate, propylene glycol monomethyl ether, methyl ethyl ketone, methyl isobutyl ketone, neopentyl acetate, butyl acetate, diethylene glycol ethyl ether, dichloromethane, and tetrahydrofuran.

9. The positive photoresist composition according to claim 8, wherein the mass of the matrix accounts for 2%-30%, preferably 4%-20%, of the total mass of the positive photoresist composition;
preferably, in the photoresist composition, the mass of the photoacid generator accounts for 2%-30%, preferably 5%-20%, of the mass of the matrix;
preferably, in the photoresist composition, the mass of the organic base accounts for 0.02%-8% of the mass of the matrix;
preferably, in the photoresist composition, the mass of the organic solvent accounts for 70%-96% of the total mass of the photoresist composition.

10. Use of the compound according to claim 1 in deep ultraviolet (248 nm and 193 nm) lithography, extreme ultraviolet (EUV) lithography, nanoimprint lithography (NIL), and electron beam lithography (EBL).
